(19) United States
(12) Patent Application Publication
(10) Pub. No.: US 2015/0297890 A1
(43) Pub. Date: Oct. 22, 2015

(54) SYSTEMS AND METHODS FOR FACILITATING USE OF A MIDDLE EAR ANALYZER IN DETERMINING ONE OR MORE STAPEDIUS REFLEX THRESHOLDS ASSOCIATED WITH A COCHLEAR IMPLANT PATIENT

(71) Applicant: ADVANCED BIONICS AG, Staefa (CH)

(72) Inventors: Anthony J. Spahr, Santa Clarita, CA (US); Leonid M. Litvak, Los Angeles, CA (US); Kinuko Masaki, San Francisco, CA (US); Charles C. Finley, Stevenson Ranch, CA (US)

(73) Assignee: Advance Bionics AG, Staefa (CH)

(21) Appl. No.: 14/648,645

(22) PCT Filed: Nov. 30, 2012
(86) PCT No.: PCT/US12/67354

§ 371 Date: May 29, 2015

(51) Int. Cl. A61N 1/36; A61N 1/02; A61N 1/05

(52) U.S. Cl. CPC A61N 1/36032; A61N 1/0541; A61N 1/025

(57) ABSTRACT

An exemplary system includes a detection facility configured to 1) receive an acoustic signal transmitted by a middle ear analyzer and 2) detect a sound level of the acoustic signal, and a processing facility configured to 1) use the sound level of the acoustic signal to control a current level of electrical stimulation applied by a cochlear implant system by way of a first set of one or more electrodes implanted within a patient, 2) synchronize the middle ear analyzer with the cochlear implant system in accordance with a first mapping data set during a first stapedius reflex measurement session, 3) identify a current level of the electrical stimulation at which the stapedius reflex occurs, and 4) automatically generate a second mapping data set based on the identified current level for use during a second stapedius reflex measurement session subsequent to the first stapedius reflex measurement session.

### BACKGROUND INFORMATION

To overcome some types of hearing loss, numerous cochlear implant systems—or cochlear prostheses—have been developed. Cochlear implant systems bypass the hair cells in the cochlea by presenting electrical stimulation directly to the auditory nerve fibers by way of one or more channels formed by an array of electrodes implanted in the cochlea. Direct stimulation of the auditory nerve fibers leads to the perception of sound in the brain and at least partial restoration of hearing function.

When a cochlear implant system is initially implanted in a patient, and during follow-up tests and checkups thereafter, it is usually necessary to fit the cochlear implant system to the patient. Such “fitting” includes adjustment of the base amplitude or intensity of the various stimuli generated by the cochlear implant system from the factory settings (or default values) to values that are most effective and comfortable for the patient. For example, the intensity or amplitude and/or duration of the individual stimulation pulses provided by the cochlear implant system may be mapped to an appropriate dynamic audio range so that the appropriate “loudness” of sensed audio signals is perceived. That is, loud sounds should be sensed by the patient at a level that is perceived as loud, but not painfully loud. Soft sounds should similarly be sensed by the patient at a level that is soft, but not so soft that the sounds are not perceived at all.

Hence, fitting and adjusting the intensity of the stimuli and other parameters of a cochlear implant system to meet a particular patient's needs requires the determination of one or more most comfortable current levels (“M levels”). An M level refers to a stimulation current level applied by a cochlear implant system at which the patient is most comfortable. M levels typically vary from patient to patient and from channel to channel in a multichannel cochlear implant.

M levels are typically determined based on subjective feedback provided by cochlear implant patients. For example, a clinician may present various stimuli to a patient and then analyze subjective feedback provided by the patient as to how the stimuli were perceived. Such subjective feedback typically takes the form of either verbal (adult) or non-verbal (child) feedback. Unfortunately, relying on subjective feedback in this manner is difficult, particularly for those patients who may have never heard sound before and/or who have never heard electrically-generated “sound.” For young children, the problem is exacerbated by a short attention span, as well as difficulty in understanding instructions and concepts, such as high and low pitch, softer and louder, same and different. Moreover, many patients, such as infants and those with multiple disabilities, are completely unable to provide subjective feedback.

Hence, it is often desirable to employ an objective method of determining M levels for a cochlear implant patient. One such objective method involves applying electrical stimulation with a cochlear implant system to a patient until a stapedius reflex (i.e., an involuntary muscle contraction that occurs in the middle ear in response to acoustic and/or electrical stimulation) is elicited. This is because the current level required to elicit a stapedius reflex within a patient (referred to herein as a “stapedius reflex threshold”) is highly correlated with (e.g., in many cases, substantially equal to) an M level corresponding to the patient. However, currently available techniques for measuring the current level at which a stapedius reflex actually occurs within a cochlear implant patient are unreliable, time consuming, and difficult to implement (especially with pediatric patients).

For example, a middle ear analyzer is often used to objectively measure a sound level at which an acoustic stimulus elicits a stapedius reflex in a non-cochlear implant patient by applying the acoustic stimulus to the ear of the non-cochlear implant patient and recording the resulting change in acoustic immittance. It would be desirable for a middle ear analyzer to be adapted for a cochlear implant patient by configuring the middle ear analyzer to record a change in acoustic immittance that occurs in response to electrical stimulation provided by the cochlear implant system. The change in the acoustic immittance could then be used to derive the stapedius reflex threshold.

However, it is currently difficult and time consuming for a clinician to use separate and unsynchronized devices to apply electrical stimulation and measure the resulting change in acoustic immittance. For example, the clinician may direct the cochlear implant system to step through a plurality of current levels as the middle ear analyzer records the resulting change in acoustic immittance. However, because the middle ear analyzer is not synchronized with the cochlear implant system (i.e., the middle ear analyzer does not “know” which current level is being applied by the cochlear implant system at any given time), it is impossible for the middle ear analyzer to correlate the recorded changes in acoustic immittance with the various current levels that are applied to the patient. Hence, the acoustic immittance recordings generated by the middle ear analyzer may be difficult or even impossible to interpret.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 illustrates an exemplary stapedius reflex elicitation and measurement system according to principles described herein.

FIG. 2 shows various components of a middle ear analyzer according to principles described herein.

FIG. 3 shows various components of an interface system according to principles described herein.

FIG. 4 shows various components of a cochlear implant system according to principles described herein.

FIG. 5 illustrates an exemplary implementation of the system of FIG. 1 according to principles described herein.

FIG. 6 illustrates an exemplary method of facilitating use of a middle ear analyzer in determining one or more stapedius reflex thresholds associated with a cochlear implant patient according to principles described herein.

FIG. 7 illustrates an exemplary computing device according to principles described herein.

### DETAILED DESCRIPTION

Systems and methods for facilitating use of a middle ear analyzer in eliciting a stapedius reflex in a cochlear implant patient and determining a stapedius reflex threshold associated with the stapedius reflex (i.e., a current level at which the stapedius reflex occurs) are described herein. For example, an exemplary system may include a detection facility and a processing facility communicatively coupled one to another. The detection facility may 1) receive an acoustic signal transmitted by a middle ear analyzer (e.g., an elicitor signal generated by the middle ear analyzer) and 2) detect a sound level of the acoustic signal. The processing facility may 1) use the sound level of the acoustic signal to control a current level of electrical stimulation applied by a cochlear implant system by way of a first set of one or more electrodes implanted within a patient, 2) synchronize the middle ear analyzer with the cochlear implant system in accordance with a first mapping data set representative of an association between a plurality of sound levels and a plurality of current levels during a first stapedius reflex measurement session in which the middle ear analyzer incrementally increases the sound level of the acoustic signal until the middle ear analyzer detects a stapedius reflex that occurs in response to the cochlear implant system applying the electrical stimulation by way of the first set of one or more electrodes, 3) identify a current level of the electrical stimulation at which the stapedius reflex occurs, and 4) automatically generate a second mapping data set based on the identified current level for use during a second stapedius reflex measurement session subsequent to the first stapedius reflex measurement session.

The systems and methods described herein may allow a middle ear analyzer to operate as it normally would (e.g., by generating an acoustic signal and increasing the sound level of the acoustic signal until a detected change in acoustic immittance indicates an occurrence of a stapedius reflex). However, instead of applying the acoustic signal directly to the patient, the acoustic signal is input into an interface system implementing the systems and methods described herein. The interface system converts the sound level and the frequency of the acoustic signal into a current level and one or more electrodes (i.e., one or more electrode numbers), respectively, and directs a cochlear implant system to apply electrical stimulation having the converted current level to one or more stimulation sites within a patient by way of the one or more electrodes. Hence, the change in acoustic immittance detected by the middle ear analyzer is actually in response to electrical stimulation representative of the acoustic signal, and not in direct response to the acoustic signal itself. However, because the operation of the middle ear analyzer and the cochlear implant system is synchronized (i.e., the cochlear implant system operates in response to and in accordance with acoustic signals provided by the middle ear analyzer), the detected change in acoustic immittance may be used to derive a stapedius reflex threshold associated with the one or more electrodes (which, as described above, may be correlated with the M levels of the one or more electrodes).

The systems and methods described herein may benefit any cochlear implant patient. For example, the systems and methods described herein may be advantageous in settings in which a pediatric patient is being fitted with a cochlear implant system. As mentioned, pediatric patients have relatively short attention spans and are often incapable of providing subjective feedback. However, because the operation of the middle ear analyzer and the cochlear implant system is synchronized, the time required to acquire one or more stapedius reflex threshold values for a pediatric patient is greatly reduced compared to conventional stapedius reflex threshold acquisition techniques.

As mentioned, the systems and methods described herein may synchronize the middle ear analyzer with the cochlear implant system during a stapedius reflex measurement session in accordance with a mapping data set. As used herein, a “mapping data set” (or simply “mapping data”) may represent an association between a plurality of sound levels and a plurality of current levels and may be used by an interface system to convert a sound level of an acoustic signal received from the middle ear analyzer into a current level to be used as the current level of electrical stimulation provided by the cochlear implant system. A “stapedius reflex measurement session” refers to a time period in which the middle ear analyzer incrementally increases the sound level of the acoustic signal until a stapedius reflex that occurs in response to the cochlear implant system applying the electrical stimulation by way of a set of one or more electrodes (e.g., a set of one or more electrodes included in an array of electrodes implanted within a patient) is detected (either automatically by the middle ear analyzer or manually by a clinician or other user). As the sound level is incrementally increased during the stapedius reflex measurement session, the interface system ensures that the middle ear analyzer and the cochlear implant system are synchronized by dynamically translating the sound level into a series of increasing current level values in accordance with the mapping data set and directing the cochlear implant system to dynamically increase the current level of the electrical stimulation being applied by way of the set of one or more electrodes in accordance with the series of increasing current level values. When the electrical stimulation elicits a stapedius reflex, the stapedius reflex measurement session is terminated.

In some examples, various mapping data sets may be automatically generated and used by the interface system (e.g., over the course of a plurality of stapedius reflex measurement sessions) to translate the detected sound levels into current levels. For example, a first mapping data set (e.g., a default mapping data set maintained by the interface system) may be used during a first stapedius reflex measurement session in which electrical stimulation is applied to a first set of one or more electrodes. The interface system may identify a current level at which a stapedius reflex occurs during the first stapedius reflex measurement session (i.e., a stapedius reflex threshold) and automatically generate a second mapping data set (e.g., a refined version of the first mapping data set) based on the identified current level. The second mapping data set may be used by the interface system to translate sound levels to current levels during a second stapedius reflex measurement session subsequent to the first stapedius reflex measurement session (e.g., during a stapedius reflex measurement session in which electrical stimulation is applied to a subset of the first set of one or more electrodes).

By automatically generating refined mapping data sets over the course of a plurality of stapedius reflex measurement sessions, the interface system may dynamically improve (e.g., make more efficient and/or accurate) the process of determining stapedius reflex thresholds associated with a plurality of electrodes implanted within a cochlear implant patient. For example, an array of sixteen electrodes (or any other number of electrodes) may be implanted within a cochlear implant patient. During an initial stapedius reflex measurement session, a default mapping data set maintained by the interface system may be used to concurrently apply electrical stimulation to all sixteen of the electrodes in accordance with a sound level of an acoustic signal provided by a middle ear analyzer. Once a stapedius reflex is elicited by the concurrent application of electrical stimulation to all sixteen electrodes, the current level used to elicit the stapedius reflex may be identified, designated as a stapedius reflex threshold corresponding to all sixteen electrodes, and used to generate a second mapping data set. The second mapping data set may be a refined version of the default mapping data set (e.g., the sound levels within the default mapping data set may be remapped to new current level values included within a more narrow range of the stapedius reflex threshold identified during the initial stapedius reflex measurement session). The second mapping data set may be used during a subsequent stapedius reflex measurement session to determine a stapedius reflex threshold associated with a subset of the sixteen electrodes (e.g., four electrodes included in electrode array). Because the range of current levels is refined in the second mapping data set compared to the initial mapping data set, the amount of time required to generate a stapedius reflex in response to electrical stimulation provided by way of the subset of electrodes may be decreased.

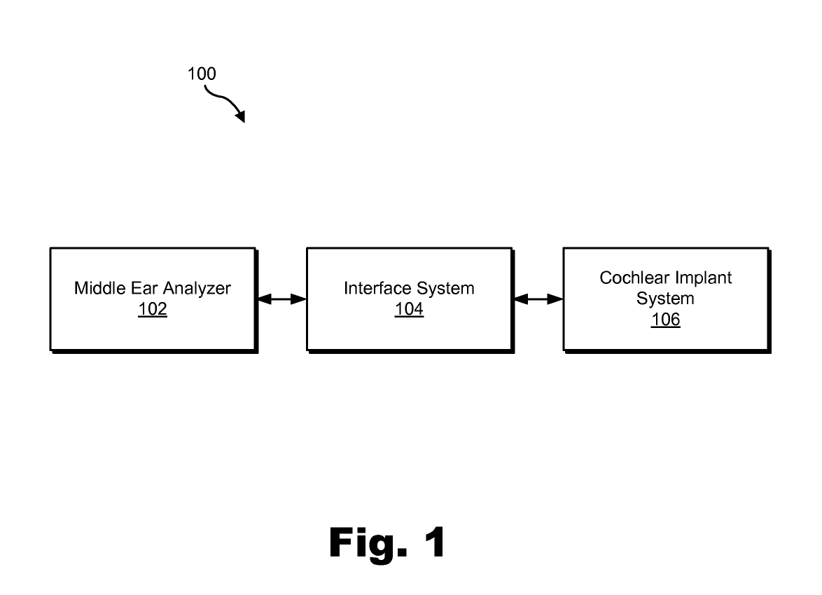

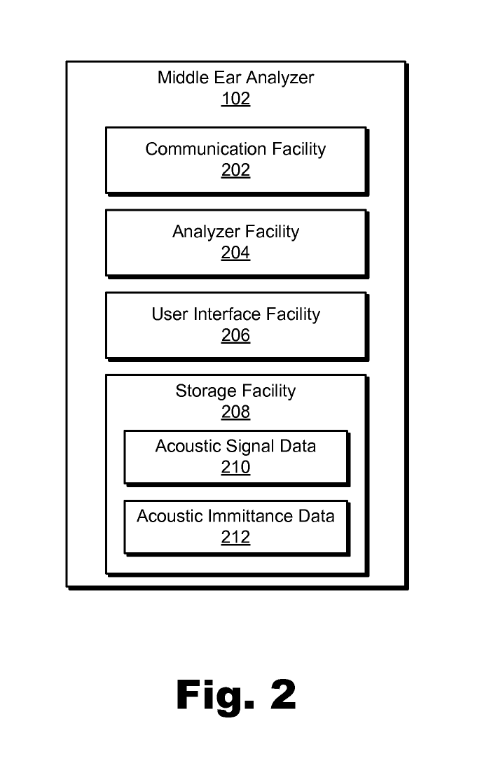

FIG. 1 illustrates an exemplary stapedius reflex elicitation and measurement system 100 (or simply “system 100”). System 100 may be configured to elicit one or more stapedius reflexes within a cochlear implant patient and identify one or more current levels at which the one or more stapedius reflexes occur (i.e., one or more stapedius reflex thresholds). To this end, system 100 may include a middle ear analyzer 102, an interface system 104, and a cochlear implant system 106 communicatively coupled to one another. Each of these components will now be described in connection with FIGS. 2-4.

FIG. 2 shows various components of middle ear analyzer 102. As shown, middle ear analyzer 102 may include, without limitation, a communication facility 202, an analyzer facility 204, a user interface facility 206, and a storage facility 208 communicatively coupled to one another. It will be recognized that although facilities 202-208 are shown to be separate facilities in FIG. 2, any of facilities 202-208 may be combined into fewer facilities, such as into a single facility, or divided into more facilities as may serve a particular implementation.

Communication facility 202 may be configured to facilitate communication between middle ear analyzer 102 and interface system 104 (e.g., by way of a probe). Communication facility 202 may additionally or alternatively be configured to facilitate data transmission between middle ear analyzer 102 and interface system 104. For example, communication facility 202 may be configured to facilitate transmission of an acoustic signal to interface system 104.

Analyzer facility 204 may be configured to perform one or more middle ear analysis functions. For example, analyzer facility 204 may be configured to generate and transmit an acoustic signal to interface system 104. As will be described in more detail below, interface system 104 may direct cochlear implant system 106 to apply electrical stimulation in accordance with the acoustic signal to one or more stimulation sites within a patient (e.g., one or more stimulation sites along an auditory pathway of the patient).

Analyzer facility 204 may be further configured to measure and record a change in acoustic immittance that occurs in response to application of the electrical stimulation applied in accordance with the acoustic signal. As used herein, “acoustic immittance” may refer to an acoustic impedance, admittance, and/or combination thereof. For example, acoustic immittance may refer to a ratio of sound pressure to volume velocity within the ear canal that occurs in response to application of electrical and/or acoustic stimulation of the auditory pathway of the patient.

In some examples, analyzer facility 204 may be configured to incrementally increase the sound level of the acoustic signal provided to interface system 104 until analyzer facility 204 (or a clinician) detects a change in the acoustic immittance that indicates an occurrence of a stapedius reflex. This may be performed in any suitable manner. For example, during a particular stapedius reflex measurement session, analyzer facility 204 may incrementally increase the sound level of the acoustic signal (e.g., step through a sequence of discrete sound levels specified in a mapping data set maintained by interface system 104) until analyzer facility 204 detects that the change in acoustic immittance reaches a predetermined threshold. In some examples, analyzer facility 204 may be configured to cease providing transmitting the acoustic signal once analyzer facility 204 determines that a stapedius reflex has occurred.

In some examples, analyzer facility 204 may be configured to stop increasing the sound level of the acoustic signal once the sound level is equal to a predetermined maximum threshold level (e.g., an “uncomfortable level” or “U level” of a cochlear implant patient) even if a stapedius reflex has not been detected. The U level of a cochlear implant patient may be determined in any suitable manner. In this manner, the patient will not be over-stimulated.

In some examples, analyzer facility 204 may be configured to set a frequency of the acoustic signal that is transmitted to interface system 104 in order to specify a set of one or more electrodes by which cochlear implant system 106 is to apply electrical stimulation. For example, a first frequency may designate a first set of one or more electrodes (e.g., electrodes one through four in an electrode array), a second frequency may designate a second set of one or more electrodes (e.g., electrodes five through eight in an electrode array), etc. It will be recognized that any combination of electrodes (e.g., all of the electrodes included in the electrode array) may be specified by the frequency of the acoustic signal provided by analyzer facility 204.

User interface facility 206 may be configured to provide one or more graphical user interfaces (“GUIs”) associated with an operation of middle ear analyzer 102. For example, a GUI may be provided and configured to facilitate user input identifying various frequencies and sound levels that the clinician desires to test with a particular patient.

Storage facility 208 may be configured to maintain acoustic signal data 210 representative of one or more acoustic signals generated by analyzer facility 204 and/or acoustic immittance data 212 representative of one or more acoustic immittance measurements made by analyzer facility 204. It will be recognized that storage facility 208 may maintain additional or alternative data as may serve a particular implementation.

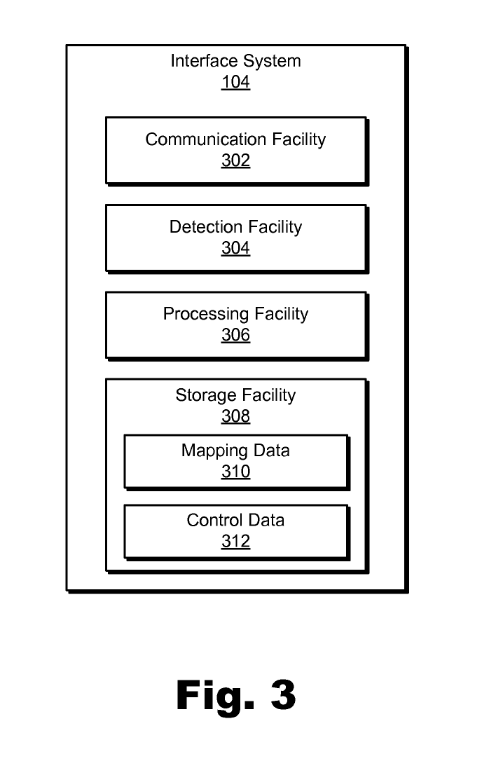

FIG. 3 shows various components of interface system 104. As shown, interface system 104 may include, without limitation, a communication facility 302, a detection facility 304, a processing facility 306, and a storage facility 308 communicatively coupled to one another. It will be recognized that although facilities 302-308 are shown to be separate facilities in FIG. 3, any of facilities 302-308 may be combined into fewer facilities, such as into a single facility, or divided into more facilities as may serve a particular implementation.

Communication facility 302 may be configured to facilitate communication between interface system 104 and middle ear analyzer 102. Communication facility 302 may be further configured to facilitate communication between interface system 104 and cochlear implant system 106. To this end, communication facility 302 may be configured to employ any suitable combination of ports, communication protocols, and data transmission means.

Detection facility 304 may be configured to receive one or more acoustic signals transmitted by a middle ear analyzer 102 and detect a sound level and frequency of the one or more acoustic signals. Detection facility 304 may employ any suitable signal processing heuristic to detect the sound level and frequency of an acoustic signal as may serve a particular implementation.

Processing facility 306 may be configured to perform any suitable processing operation related to one or more acoustic signals detected by detection 304. For example, processing facility 306 may be configured to manage (e.g., maintain, generate, update, etc.) mapping data representative of an association between a plurality of sound levels and a plurality of current levels and between a plurality of frequencies and a plurality of electrodes. Mapping data may be maintained in the form of a look-up table, in a database, and/or in any other manner as may serve a particular implementation.

To illustrate, Table 1 illustrates a mapping data set representative of an exemplary association between a plurality of sound levels and a plurality of current levels that may be maintained by processing facility 306.

TABLE 1

| Sound Level | Current Level |
|---|---|
| (dB SPL) | (CU) |
| 80 | 110 |
| 85 | 120 |
| 90 | 130 |
| 95 | 140 |
| 100 | 150 |

As shown in Table 1, the mapping data set indicates that a sound level of 80 dB SPL is mapped to a current level of 110 clinical units (“CU”), a sound level of 85 dB SPL is mapped to a current level of 120 CU, a sound level of 90 dB SPL is mapped to a current level of 130 CU, a sound level of 95 dB SPL is mapped to a current level of 140 CU, and a sound level of 100 dB SPL is mapped to a current level of 150 CU. As will be described below, processing facility 306 may use a mapping data set similar to that illustrated in Table 1 to identify a current level that is associated with a sound level of a particular acoustic signal detected by detection facility 304. It will be recognized that the mapping data set illustrated in Table 1 is merely illustrative of the many different mapping data sets that may be utilized and/or generated in accordance with the systems and methods described herein. In some examples, processing facility 306 may interpolate between the various data points included in Table 1 to determine the relationship between sound level and current level for values not specifically included in Table 1. For example, processing facility 306 may use one or more interpolation techniques to determine a current level that corresponds to a sound level of 82 dB SPL, even though this particular sound level is not specifically included in Table 1. Moreover, it will be recognized in some alternative embodiments, an equation may be used to define the relationship between sound level and current level.

In some examples, processing facility 306 may maintain a default mapping data set representative of an association between a plurality of sound levels and a plurality of current levels. The default mapping data set may be used during an initial stapedius reflex measurement session, for example, to identify a current level at which a stapedius reflex occurs (i.e., a stapedius reflex threshold) when electrical stimulation is presented by cochlear implant system 106 by way of a particular set of one or more electrodes. As will be described in more detail below, processing facility 306 may use the identified current level to generate a refined mapping data set for use during one or more subsequent stapedius reflex measurement sessions.

Table 2 illustrates an exemplary mapping data set representative of an exemplary association between a plurality of frequencies and a plurality of electrodes (e.g., a plurality of electrodes included in an array of electrodes configured to be implanted within a cochlea of a patient) that may be maintained by processing facility 306.

TABLE 2

| Frequency | Electrode |
|---|---|
| (kHz) | Numbers |
| 1 | 1-4 |
| 2 | 5-8 |
| 3 | 9-12 |
| 4 | 13-16 |
| 5 | 1-16 |

As shown in Table 2, the additional mapping data indicates that a frequency of 1 kHz is mapped to electrodes 1 through 4, a frequency of 2 kHz is mapped to electrodes 5 through 8, a frequency of 3 kHz is mapped to electrodes 9 through 12, a frequency of 4 kHz is mapped to electrodes 13 through 16, and a frequency of 5 kHz is mapped to electrodes 1 through 16. Other combinations of electrodes may be represented by other frequencies as may serve a particular implementation. As will be described below, processing facility 306 may use the mapping data set illustrated in Table 2 to identify one or more electrodes that are associated with a frequency of a particular acoustic signal detected by detection facility 304. It will be recognized that the mapping associations between frequency and electrode numbers illustrated in Table 2 are merely illustrative of the many different mapping associations that may be utilized in accordance with the systems and methods described herein.

Processing facility 306 may be further configured to facilitate elicitation and measurement of a stapedius reflex during a stapedius reflex measurement session. For example, an acoustic signal may be transmitted to interface system 104 by middle ear analyzer 102 during a particular stapedius reflex measurement session. Processing facility 306 may utilize the mapping data described above to identify a current level that corresponds to the sound level of the acoustic signal and a set of one or more electrodes that corresponds to the frequency of the acoustic signal. Processing facility 306 may then direct cochlear implant system 106 to apply electrical stimulation having the identified current level by way of the identified set of one or more electrodes.

Processing facility 306 may be further configured to synchronize the middle ear analyzer with the cochlear implant system during a stapedius reflex measurement session. In other words, processing facility 306 may ensure that the current level of the electrical stimulation being provided by cochlear implant system 106 is correlated with the sound level of the acoustic signal as the sound level of the acoustic signal is incrementally increased during the stapedius reflex measurement session.

In some examples, processing facility 306 may synchronize middle ear analyzer 102 and cochlear implant system 106 during the stapedius reflex measurement session in accordance with a mapping data set (e.g., the mapping data set illustrated in Table 1). For example, processing facility 306 may synchronize middle ear analyzer 102 and cochlear implant system 106 during the stapedius reflex measurement session by dynamically translating the sound level of the acoustic signal into a series of increasing current level values in accordance with a mapping data set as the sound level incrementally increases during the stapedius reflex measurement session and directing cochlear implant system 106 to dynamically increase the current level of the electrical stimulation being applied by way of the set of one or more electrodes in accordance with the series of increasing current level values (e.g., by transmitting one or more control parameters to cochlear implant system 106). To illustrate, middle ear analyzer 102 may incrementally step through the various sound levels included in Table 1 until a stapedius reflex is elicited. As middle ear analyzer 102 incrementally steps through the various sound levels, processing facility 306 may direct cochlear implant system 106 to incrementally increase the current level of the electrical stimulation being applied by cochlear implant system 106 in accordance with the current level values included in Table 1.

Once a stapedius reflex has been detected by middle ear analyzer 102, processing facility 306 may identify a current level of the electrical stimulation at which the stapedius reflex occurs. This may be performed in any suitable manner. For example, processing facility 306 may identify the current level by identifying the last current level used before the stapedius reflex measurement session is terminated. In some examples, processing facility 306 may designate the identified current level as a stapedius reflects threshold associated with the set of one or more electrodes by which electrical stimulation is being applied during the stapedius reflex measurement session.

Processing facility 306 may be further configured to automatically generate a new mapping data set (i.e., a new mapping data set representative of an association between a plurality of sound levels and a plurality of current levels) based on the identified current level for use during a subsequent stapedius reflex measurement session. The new mapping data set may be generated in any suitable manner.

For example, a first mapping data set (e.g., a default mapping data set or any other mapping data set generated by processing facility 306) may be used by processing facility 306 during a first stapedius reflex measurement session in which a first current level is identified as being the current level at which the stapedius reflex occurs during the first stapedius reflex measurement session. Processing facility 306 may generate a second mapping data set based on the first current level for use during a second stapedius reflex measurement session subsequent to the first stapedius reflex measurement session. The second mapping data set may be generated as a function of the first current level. For example, the sound levels within the first mapping data set may be remapped to new current level values included within a more narrow range of the first current level.

To illustrate, it will be assumed that the first mapping data set is identical to the mapping data set illustrated in Table 1 above. It will also be assumed that the current level at which the stapedius reflex occurs during the first stapedius reflex measurement session is 140 CU. Processing facility 306 may be configured to remap the current levels shown in Table 1 to a more narrow range surrounding a current level of 140 CU. For example, processing facility 306 may be configured to remap the current level shown in Table 1 to current level values included within a range of ten CU surrounding 140 CU. Such a remapping is illustrated in the second mapping data set shown in Table 3:

TABLE 3

| Sound Level | Current Level |
|---|---|
| (dB SPL) | (CU) |
| 80 | 135 |
| 85 | 137.5 |
| 90 | 140 |
| 95 | 142.5 |
| 100 | 145 |

As shown in Table 3, the second mapping data set indicates that the sound level of 80 dB SPL is now mapped to a current level of 135 CU, the sound level of 85 dB SPL is mapped to a current level of 137.5 CU the sound level of 90 dB SPL is mapped to a current level of 140 CU, the sound level of 95 dB SPL is mapped to a current level of 142.5 CU, and the sound level of 100 dB SPL is mapped to a current level of 145 CU. It will be recognized that processing facility 306 may generate the second mapping data set in any other suitable manner as may serve a particular implementation.

By automatically generating one or more mapping data sets has described above, processing facility 306 may obviate the need for a clinician to manually create a mapping data set for use during a stapedius reflex measurement session. This may result in a more efficient and accurate fitting process. Additionally or alternatively, once a particular mapping data set has been automatically generated by processing facility 306, a clinician may choose to use the newly generated mapping data set to manually determine when a stapedius reflex has occurred. For example, the clinician may manually increase the sound level of an acoustic signal provided by middle ear analyzer 102. Processing facility 306 may automatically convert the sound level to current level in accordance with the automatically generated mapping data set. The clinician may then determine when a stapedius reflex occurs in response to stimulation provided by cochlear implant system 106 (e.g., by viewing a graph generated by processing facility 306 that represents a change in acoustic immittance that occurs as the sound level is increased).

Processing facility 306 may be further configured to use the newly created mapping data set during one or more subsequent stapedius reflex measurement sessions. For example, processing facility 306 may use the second mapping data set shown in Table 3 during a second stapedius reflex measurement session that follows the first stapedius reflex measurement session.

To illustrate, middle ear analyzer 102 may transmit a second acoustic signal during the second stapedius reflex measurement session. Detection facility 304 may receive the second acoustic signal and detect a sound level and frequency of the second acoustic signal. Processing facility 306 may use the second mapping data set to identify a current level that corresponds to the sound level of the second acoustic signal and a second set of one or more electrodes that corresponds to the frequency of the second acoustic signal and control cochlear implant system 106 accordingly. During the second stapedius reflex measurement session, processing facility 306 may synchronize middle ear analyzer 102 with cochlear implant system 106 in accordance with the second mapping data set in a manner similar to that described above.

A third mapping data set may be generated during the second stapedius reflex measurement session for use during a third stapedius reflex measurement session in a manner similar to that described above. It will be recognized that any number of mapping data sets may be generated during a sequence of stapedius reflect measurement sessions as may serve a particular implementation.

In some examples, a mapping data set generated during a first stapedius reflex measurement session may be used during any subsequent stapedius reflex measurement session as may serve a particular implementation. For example, the mapping data set may be used during a stapedius reflex measurement session that immediately follows the stapedius reflex measurement session. Alternatively, the stapedius reflex measurement session during which the mapping data set is used may be temporally separated from the first stapedius reflex measurement session by at least one other intervening stapedius reflex measurement session.

To illustrate, a mapping data set may be generated during a first stapedius reflex measurement session in which electrical stimulation is applied to all sixteen electrodes in a sixteen electrode array (or all electrodes in any other size of electrode array). The mapping data set may be subsequently used during multiple stapedius reflex measurement sessions in which electrical stimulation is applied to various different subsets of the sixteen electrode array. For example, the mapping data set may be used during a second stapedius reflex measurement session in which electrical stimulation is applied to electrodes one through four of the sixteen electrode array, during a third stapedius reflex measurement session in which electrical stimulation is applied to electrodes five through eight of the sixteen electrode array, during a fourth stapedius reflex measurement session in which electrical stimulation is applied to electrodes nine through twelve, and during a fifth stapedius reflex measurement session in which electrical stimulation is applied to electrodes thirteen through sixteen.

In some examples, one or more intervening stapedius reflex measurement sessions may be temporally interspersed between the first, second, third, fourth, and fifth stapedius reflex measurement sessions described above. For example, a stapedius reflex measurement session may be performed in between the second and third stapedius reflex measurement sessions. In this stapedius reflex measurement session, a mapping data set generated during the second stapedius reflex measuring session (i.e., during the stapedius reflex measurement session in which the electrical stimulation is applied to electrodes one through four of the sixteen electrode array) may be used to determine stapedius reflex thresholds associated with electrodes one and two of the sixteen electrode array.

As mentioned, the frequency of the acoustic signal provided by middle ear analyzer 102 during any stapedius reflex measurement session may be representative of any set of one or more electrodes as may serve a particular implementation. For example, electrical stimulation may be applied by way of a first set of one or more electrodes during a first stapedius reflex measurement session and by way of a second set of one or more electrodes during a second stapedius reflex measurement session subsequent to the first stapedius reflex measurement session. The first and second sets of one or more electrodes may each include a number of electrodes as may serve a particular implementation.

To illustrate, the second set of one or more electrodes may include a subset of the first set of one or more electrodes. For example, the first set of one or more electrodes may include all of the electrodes included in a sixteen electrode array and the second set of one or more electrodes may include a set of four electrodes included in the sixteen electrode array. Alternatively, the first and second sets of one or more electrodes may be identical (e.g., the first and second sets of one or more electrodes may each include electrodes one through four of a sixteen electrode array). In yet another alternative embodiment, the second set of one or more electrodes may include at least one electrode not included in the first set of one or more electrodes. For example, the first set of one or more electrodes may include electrodes one through four of the sixteen electrode array while the second set of one or more electrodes may include electrodes five through eight of the sixteen electrode array.

In some examples, processing facility 306 may be configured to prevent cochlear implant system 106 from increasing the current level of the electrical stimulation applied to the patient beyond a U level associated with the patient. As mentioned, the U level represents an “uncomfortable level” associated with the patient. Stimulation above the U level may result in discomfort, pain, and/or damage to the patient. Hence, limiting cochlear implant system 106 from increasing the current level beyond the U level of a patient may ensure patient comfort and safety.

Processing facility 306 may be further configured to present one or more GUIs and receive user input by way of the one or more GUIs. For example, processing facility 306 may be configured to detect an occurrence of a stapedius reflex and designate the current level associated with the stapedius reflex as being an M level associated with the patient. The detection of the occurrence of the stapedius reflex may be performed automatically by processing facility 306 or in response to user input provided by way of one or more GUIs presented by processing facility 306. For example, processing facility 306 may receive user input representative of a sound level at which a stapedius reflex occurred during the application of electrical stimulation by cochlear implant system 106. Based on the user input and on the mapping data, processing facility 306 may determine a current level at which the stapedius reflex occurred, designate the current level as an M level associated with the patient, and present data representative of the M level within a GUI.

As another example, processing facility 306 may present a GUI configured to facilitate user customization of a default mapping data set maintained by processing facility 306. For example, processing facility 306 may present a GUI configured to allow a user to edit the mapping data illustrated in Table 1 and/or Table 2 as shown above. In this manner, a clinician may modify one or more mapping associations as may serve a particular implementation.

Processing facility 306 may be further configured to perform one or more calibration operations associated with a particular middle ear analyzer. For example, interface system 104 may be used in connection with a variety of different middle ear analyzers. Each middle ear analyzer may be calibrated upon being connected to interface system 104 so that appropriate current levels are applied to the patient.

In some alternative examples, it may be desirable for a user of interface system 104 to specify a particular group of electrodes to be tested (i.e., a group of electrodes for which a stapedius reflex threshold is to be determined). For example, a clinician may desire to determine the M level for a single electrode. To this end, processing facility 306 may provide a GUI configured to facilitate identification by a user of one or more specific electrodes. In response to receiving this user input, processing facility 306 may direct middle ear analyzer 102 to provide an acoustic signal having a frequency associated with the identified one or more electrodes. Detection facility 304 may detect the sound level of an acoustic signal, and processing facility 306 may identify a current level associated with the sound level based on the mapping data. Processing facility 306 may then direct a cochlear implant system to apply electrical stimulation having the identified current level by way of the identified one or more electrodes.

Storage facility 308 may be configured to maintain mapping data 310 (e.g., one or more mapping data sets) managed by processing facility 306 and control data 312 (e.g., one or more control parameters) generated by processing facility 306. It will be recognized that storage facility 308 may maintain additional or alternative data as may serve a particular implementation.

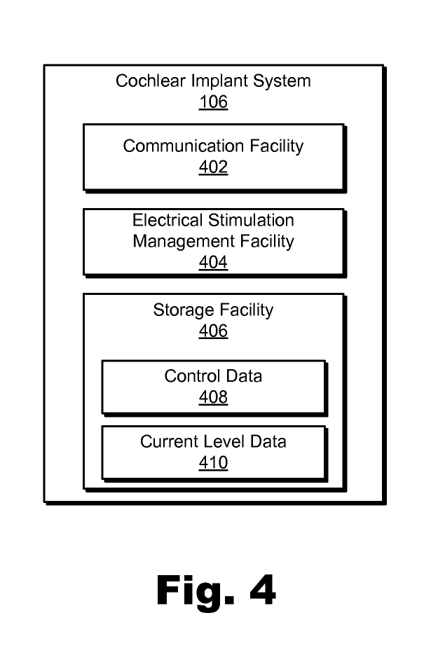

FIG. 4 shows various components of cochlear implant system 106. As shown, cochlear implant system 106 and may include, without limitation, a communication facility 402, an electrical stimulation management facility 404, and a storage facility 406 communicatively coupled to one another. It will be recognized that although facilities 402-406 are shown to be separate facilities in FIG. 4, any of facilities 402-406 may be combined into fewer facilities, such as into a single facility, or divided into more facilities as may serve a particular implementation.

Communication facility 402 may be configured to facilitate communication between cochlear implant system 106 and interface system 104. To this end, communication facility 402 may be configured to employ any suitable combination of ports, communication protocols (e.g., wired and/or wireless communication protocols), and data transmission means.

Electrical stimulation management facility 404 may be configured to perform any suitable electrical stimulation operation as may serve a particular implementation. For example, electrical stimulation management facility 404 may receive control data representative of a particular current level and one or more electrodes from interface system 104. Based on this control data, electrical stimulation management facility 404 may generate and apply electrical stimulation having the particular current level to one or more stimulation sites within a cochlear implant patient by way of the one or more electrodes. The electrical stimulation may be generated and applied in any suitable manner as may serve a particular implementation. For example, a sound processor located external to the patient may use the control data to generate one or more stimulation parameters configured to direct a cochlear implant implanted within the patient to generate and apply the electrical stimulation.

In some examples, electrical stimulation management facility 404 may incrementally increase the current level of the electrical stimulation in response to middle ear analyzer 102 incrementally increasing a sound level of an acoustic signal until a stapedius reflex that occurs in response to the electrical stimulation is detected. Once a stapedius reflex occurs, electrical stimulation management facility 404 may identify the current level at which the stapedius reflex occurs and direct storage facility 406 to store data representative of the current level. Electrical stimulation management facility 404 may then utilize the stored data to determine one or more M levels associated with the set of one or more electrodes for use in one or more stimulation programs (i.e., one or more stimulation programs used by cochlear implant system 106 during a normal operation subsequent to a fitting session in which cochlear implant system 106 is coupled to interface system 104). For example, electrical stimulation management facility 404 may use the stored data to generate and apply electrical stimulation having a current level substantially equal to the determined M levels. Electrical stimulation management facility 404 may then generate one or more user-selectable stimulation programs that utilize the generated M levels.

Storage facility 406 may be configured to maintain control data 408 received from interface system 104 and current level data 410 representative of one or more current levels at which one or more stapedius reflexes occur. It will be recognized that storage facility 406 may maintain additional or alternative data as may serve a particular implementation.

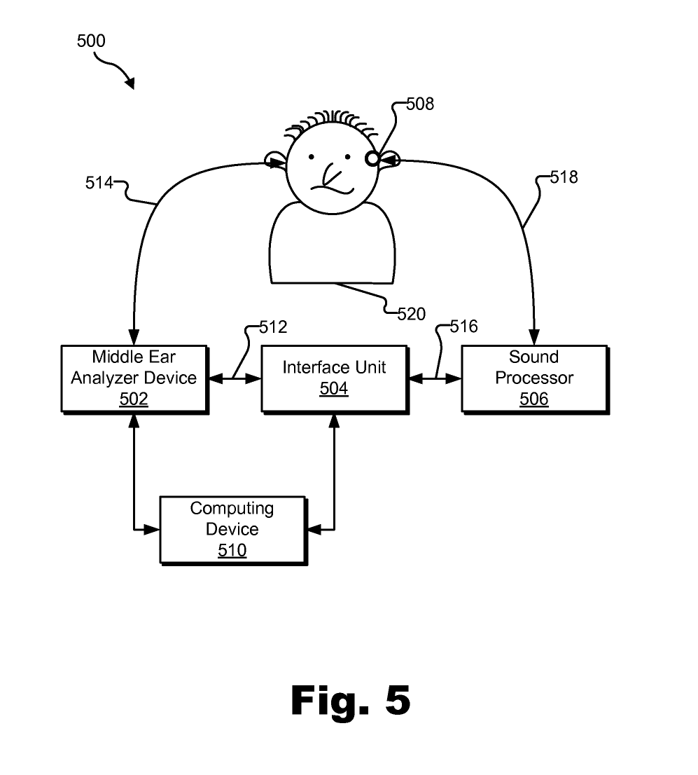

FIG. 5 illustrates an exemplary implementation 500 of system 100. As shown, implementation 500 may include a middle ear analyzer device 502, an interface unit 504, a sound processor 506, a cochlear implant 508, and a computing device 510. Implementation 500 may further include a stimulation probe 512 configured to communicatively couple middle ear analyzer device 502 and interface unit 504 and a detection probe 514 configured to be coupled to middle ear analyzer device 502 and detect a change in immittance that occurs as a result of electrical stimulation applied by way of one or more electrodes (not shown) communicatively coupled to cochlear implant 508.

Middle ear analyzer 102, interface system 104, and cochlear implant system 106 may each be implemented by one or more components illustrated in FIG. 5. For example, middle ear analyzer 102 may be implemented by middle ear analyzer device 502, stimulation probe 512, detection probe 514, and computing device 510. Interface system 104 may be implemented by interface unit 504 and computing device 510. Cochlear implant system 106 may be implemented by sound processor 506 and cochlear implant 508.

Each of the components shown in FIG. 5 will now be described in more detail. Middle ear analyzer device 502 may include any suitable middle ear analyzer (e.g. an off-the-shelf middle ear analyzer) configured to perform one or more of the middle ear analyzer operations described herein. For example, middle ear analyzer device 502 may be configured to operate in a contralateral stimulation mode in which middle ear analyzer device 502 is configured to generate and apply acoustic stimulation (i.e., one or more acoustic signals) by way of stimulation probe 512 and record a resulting change in immittance using detection probe 514.

Interface unit 504 may be configured to perform one or more interface operations as described herein. For example, interface unit 504 may include any combination of signal receivers, signal transmitters, processors, and/or computing devices configured to receive an acoustic signal transmitted by a middle ear analyzer device 502 by way of stimulation probe 512, detect a sound level and frequency of the acoustic signal, and transmit control data representative of a current level associated with the sound level and one or more electrodes associated with the frequency to sound processor 506.

Interface unit 504 may be coupled directly to middle ear analyzer device 502 by way of stimulation probe 512. Interface unit 504 may also be coupled to sound processor 506 by way of communication channel 516, which may include any suitable wired and/or wireless communication channel as may serve a particular implementation.

Sound processor 506 may include any type of sound processor used in a cochlear implant system as may serve a particular implementation. For example, sound processor 506 may include a behind-the-ear (“BTE”) sound processing unit, a portable speech processor (“PSP”), and/or a body-worn processor.

Cochlear implant 508 may include any suitable auditory prosthesis configured to be at least partially implanted within a patient as may serve a particular implementation. For example, cochlear implant 508 may include an implantable cochlear stimulator, a brainstem implant and/or any other type of auditory prosthesis. In some examples, cochlear implant 508 may be communicatively coupled to a lead having a plurality of electrodes (e.g., sixteen electrodes) disposed thereon. The lead may be configured to be implanted within the patient such that the electrodes are in communication with stimulation sites (e.g., locations within the cochlea) within the patient. As used herein, the term “in communication with” refers to an electrode being adjacent to, in the general vicinity of, in close proximity to, directly next to, or directly on a stimulation site.

Sound processor 506 and cochlear implant 508 may communicate by way of communication channel 518. Communication channel 518 may be wired or wireless as may serve a particular implementation.

Computing device 510 may include any combination of computing devices (e.g., personal computers, mobile computing devices (e.g., mobile phones, tablet computers, laptop computers, etc.), fitting stations, etc.). As shown, computing device 510 may be communicatively coupled (e.g., with one or more cables) to both the middle ear analyzer device 502 and the interface unit 504. As such, computing device 510 may be configured to perform one or more of the operations associated with the middle ear analyzer device 502 and the interface unit 504. For example, computing device 510 may generate and present one or more GUIs by way of a display device (e.g., a display screen included within computing device 510 and/or communicatively coupled to computing device 510) associated with an operation of middle ear analyzer device 502 and/or interface unit 504.

Additionally or alternatively, computing device 510 may be configured to store, maintain, process, and/or otherwise maintain the mapping data utilized by interface system 104. For example, computing device 510 may be configured to maintain a database comprising the mapping data and identify current levels and/or electrodes associated with an acoustic signal received by interface unit 504.

In some alternative examples, separate computing devices may be associated with middle ear analyzer device 502 and interface unit 504. For example, a first computing device may be communicatively coupled to middle ear analyzer device 502 and configured to perform one or more operations associated with middle ear analyzer device 502 and a second computing device may be communicatively coupled to interface unit 504 and configured to perform one or more operations associated with interface unit 504.

In yet another alternative example, interface unit 504 may not be coupled to computing device 510 or to any other computing device. In this example, interface unit 504 may be configured to perform all of the operations associated with interface system 104 as described herein.

In an exemplary configuration, detection probe 514 is placed within one of the ears of a patient 520. In some examples, as shown in FIG. 5, the ear in which detection probe 514 is placed is contralateral to the ear associated with cochlear implant 508. Alternatively, detection probe 514 may be placed within the same (i.e., ipsilateral) ear associated with cochlear implant 508.

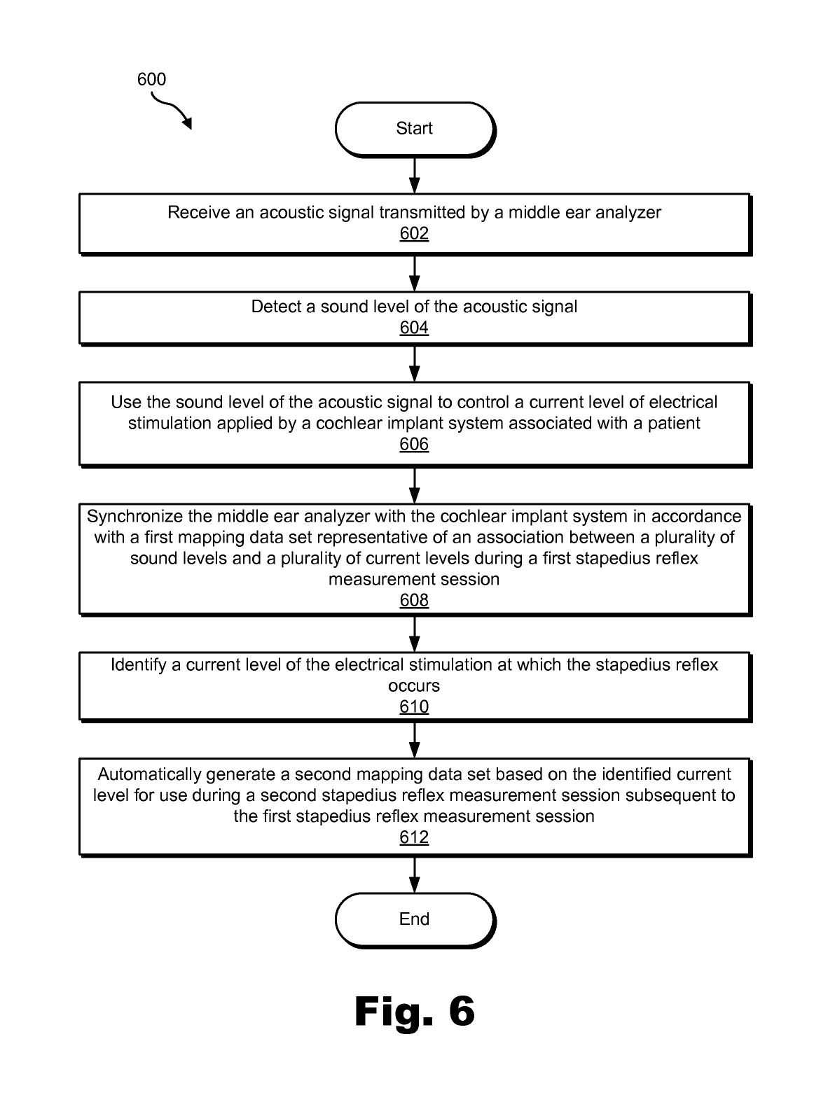

FIG. 6 illustrates an exemplary method 600 of facilitating use of a middle ear analyzer in determining one or more stapedius reflex thresholds associated with a cochlear implant patient. While FIG. 6 illustrates exemplary steps according to one embodiment, other embodiments may omit, add to, reorder, and/or modify any of the steps shown in FIG. 6. One or more of the steps shown in FIG. 6 may be performed by interface system 104 and/or any implementation thereof.

In step 602, an interface system receives an acoustic signal transmitted by a middle ear analyzer. Step 602 may be performed in any of the ways described herein.

In step 604, the interface system detects a sound level of the acoustic signal. Step 604 may be performed in any of the ways described herein.

In step 606, the interface system uses the sound level of the acoustic signal to control a current level of electrical stimulation applied by a cochlear implant system associated with a patient. Step 606 may be performed in any of the ways described herein.

In step 608, the interface system synchronizes the middle ear analyzer with the cochlear implant system in accordance with a first mapping data set representative of an association between a plurality of sound levels and a plurality of current levels during a first stapedius reflex measurement session. As described above, during the first stapedius reflex measurement session, the middle ear analyzer incrementally increases the sound level of the acoustic signal until a stapedius reflex that occurs in response to the cochlear implant system applying the electrical stimulation by way of a first set of one or more electrodes implanted within the patient is detected (e.g., automatically by the middle ear analyzer or manually by a clinician). Step 608 may be performed in any of the ways described herein.

In step 610, the interface system identifies a current level of the electrical stimulation at which the stapedius reflex occurs. Step 610 may be performed in any of the ways described herein.

In step 612, the interface system automatically generates a second mapping data set based on the identified current level for use during a second stapedius reflex measurement session subsequent to the first stapedius reflex measurement session. Step 612 may be performed in any of the ways described herein.

An example of the systems and methods described herein will now be provided. It will be recognized that this example is merely illustrative of the many different implementations that may be realized in accordance with the systems and methods described herein.

In this example, a clinician may desire to determine a plurality of M levels associated with a cochlear implant patient (e.g., a pediatric cochlear implant patient). To this end, the clinician may utilize the configuration shown in FIG. 5. For example, the clinician may program middle ear analyzer 502 to operate in a contralateral stimulation mode, place probe 514 within one of the ears of the patient, and ensure that probe 512 is connected to interface unit 504 and that interface unit 504 is in turn connected to sound processor 506. The clinician may then initiate stapedius reflex measurement session (e.g., by pressing “start” on middle ear analyzer 502). In response, middle ear analyzer 502 may automatically generate and transmit acoustic signals to interface unit 504 in accordance with a default mapping data set. Interface unit 504 (and, in some implementations, computing device 510) may direct sound processor 506 and cochlear implant 508 to generate and apply electrical stimulation representative of the acoustic signals to the patient. Middle ear analyzer 502 may record the various changes in acoustic immittance that occur as a result of the electrical stimulation and automatically determine that a stapedius reflex has occurred in response to the electrical stimulation. Middle ear analyzer 502 may then (e.g., automatically) proceed to perform one or more additional stapedius reflex measurement session until a desired number of stapedius reflexes have been detected.

In certain embodiments, one or more of the processes described herein may be implemented at least in part as instructions embodied in a non-transitory computer-readable medium and executable by one or more computing devices. In general, a processor (e.g., a microprocessor) receives instructions, from a non-transitory computer-readable medium, (e.g., a memory, etc.), and executes those instructions, thereby performing one or more processes, including one or more of the processes described herein. Such instructions may be stored and/or transmitted using any of a variety of known computer-readable media.

A computer-readable medium (also referred to as a processor-readable medium) includes any non-transitory medium that participates in providing data (e.g., instructions) that may be read by a computer (e.g., by a processor of a computer). Such a medium may take many forms, including, but not limited to, non-volatile media, and/or volatile media. Non-volatile media may include, for example, optical or magnetic disks and other persistent memory. Volatile media may include, for example, dynamic random access memory (“DRAM”), which typically constitutes a main memory. Common forms of computer-readable media include, for example, a disk, hard disk, magnetic tape, any other magnetic medium, a CD-ROM, DVD, any other optical medium, a RAM, a PROM, an EPROM, a FLASH-EEPROM, any other memory chip or cartridge, or any other tangible medium from which a computer can read.

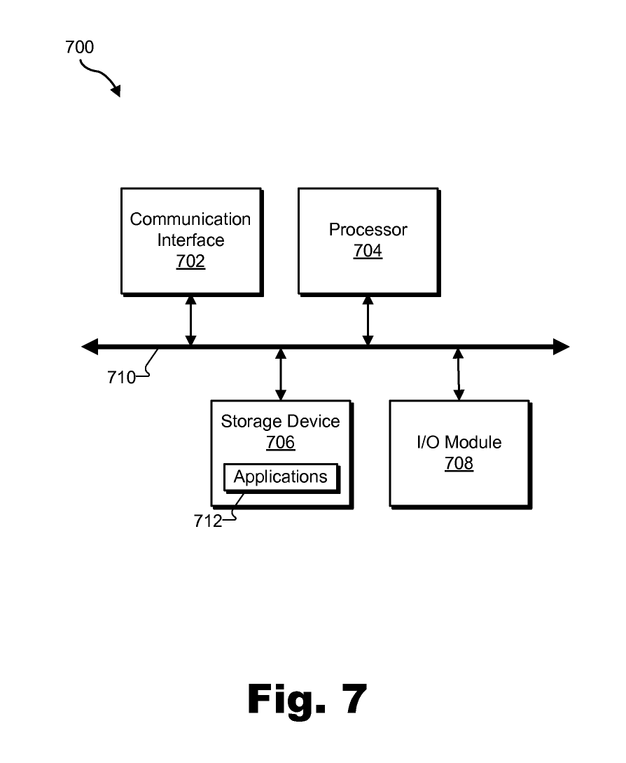

FIG. 7 illustrates an exemplary computing device 700 that may be configured to perform one or more of the processes described herein. As shown in FIG. 7, computing device 700 may include a communication interface 702, a processor 704, a storage device 706, and an input/output (“I/O”) module 708 communicatively connected via a communication infrastructure 710. While an exemplary computing device 700 is shown in FIG. 7, the components illustrated in FIG. 7 are not intended to be limiting. Additional or alternative components may be used in other embodiments. Components of computing device 700 shown in FIG. 7 will now be described in additional detail.

Communication interface 702 may be configured to communicate with one or more computing devices. Examples of communication interface 702 include, without limitation, a wired network interface (such as a network interface card), a wireless network interface (such as a wireless network interface card), a modem, an audio/video connection, and any other suitable interface.

Processor 704 generally represents any type or form of processing unit capable of processing data or interpreting, executing, and/or directing execution of one or more of the instructions, processes, and/or operations described herein. Processor 704 may direct execution of operations in accordance with one or more applications 712 or other computer-executable instructions such as may be stored in storage device 706 or another computer-readable medium.

Storage device 706 may include one or more data storage media, devices, or configurations and may employ any type, form, and combination of data storage media and/or device. For example, storage device 706 may include, but is not limited to, a hard drive, network drive, flash drive, magnetic disc, optical disc, random access memory (“RAM”), dynamic RAM (“DRAM”), other non-volatile and/or volatile data storage units, or a combination or sub-combination thereof. Electronic data, including data described herein, may be temporarily and/or permanently stored in storage device 706. For example, data representative of one or more executable applications 712 configured to direct processor 704 to perform any of the operations described herein may be stored within storage device 706. In some examples, data may be arranged in one or more databases residing within storage device 706.

I/O module 708 may be configured to receive user input and provide user output and may include any hardware, firmware, software, or combination thereof supportive of input and output capabilities. For example, I/O module 708 may include hardware and/or software for capturing user input, including, but not limited to, a keyboard or keypad, a touch screen component (e.g., touch screen display), a receiver (e.g., an RF or infrared receiver), and/or one or more input buttons.

I/O module 708 may include one or more devices for presenting output to a user, including, but not limited to, a graphics engine, a display (e.g., a display screen, one or more output drivers (e.g., display drivers), one or more audio speakers, and one or more audio drivers. In certain embodiments, I/O module 708 is configured to provide graphical data to a display for presentation to a user. The graphical data may be representative of one or more graphical user interfaces and/or any other graphical content as may serve a particular implementation.

In some examples, any of the facilities described herein may be implemented by or within one or more components of computing device 700. For example, one or more applications 712 residing within storage device 706 may be configured to direct processor 704 to perform one or more processes or functions associated with middle ear analyzer 102, interface system 104, and/or cochlear implant system 106.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

What is claimed is:

1. A system comprising:
a detection facility configured to
receive an acoustic signal transmitted by a middle ear analyzer, and
detect a sound level of the acoustic signal; and
a processing facility communicatively coupled to the detection facility and configured to
use the sound level of the acoustic signal to control a current level of electrical stimulation applied by a cochlear implant system by way of a first set of one or more electrodes implanted within a patient,
synchronize the middle ear analyzer with the cochlear implant system in accordance with a first mapping data set representative of an association between a plurality of sound levels and a plurality of current levels during a first stapedius reflex measurement session in which the middle ear analyzer incrementally increases the sound level of the acoustic signal until a stapedius reflex that occurs in response to the cochlear implant system applying the electrical stimulation by way of the first set of one or more electrodes is detected,
identify a current level of the electrical stimulation at which the stapedius reflex occurs, and
automatically generate a second mapping data set based on the identified current level for use during a second stapedius reflex measurement session subsequent to the first stapedius reflex measurement session.

2. The system of claim 1, wherein the processing facility is configured to synchronize the middle ear analyzer with the cochlear implant system in accordance with the first mapping data set during the first stapedius reflex measurement session by:
dynamically translating the sound level into a series of increasing current level values in accordance with the first mapping data set as the sound level incrementally increases during the first stapedius reflex measurement session; and
directing the cochlear implant system to dynamically increase the current level of the electrical stimulation being applied by way of the first set of one or more electrodes in accordance with the series of increasing current level values as the sound level incrementally increases during the first stapedius reflex measurement session.

3. The system of claim 1, wherein:
the second mapping data set is representative of an association between the plurality of sound levels and another plurality of current levels; and
the detection facility is further configured to
receive an additional acoustic signal transmitted by the middle ear analyzer during the second stapedius reflex measurement session, and
detect a sound level of the additional acoustic signal; and
the processing facility is further configured to
use the sound level of the additional acoustic signal to control a current level of electrical stimulation applied by the cochlear implant system by way of a second set of electrodes during the second stapedius reflex measurement session; and
synchronize the middle ear analyzer with the cochlear implant system in accordance with the second mapping data set during the second stapedius reflex measurement session in which the middle ear analyzer incrementally increases the sound level of the additional acoustic signal until a stapedius reflex that occurs in response to the cochlear implant system applying the electrical stimulation during the second stapedius reflex measurement session is detected.

4. The system of claim 3, wherein the processing facility is further configured to:
identify a current level at which the stapedius reflex occurs in response to the cochlear implant system applying the electrical stimulation during the second stapedius reflex measurement session; and
automatically generate a third mapping data set based on the identified current level at which the stapedius reflex occurs in response to the cochlear implant system applying the electrical stimulation during the second stapedius reflex measurement session for use during a third stapedius reflex measurement session subsequent to the second stapedius reflex measurement session.

5. The system of claim 3, wherein the second set of one or more electrodes comprises a subset of the first set of one or more electrodes.

6. The system of claim 3, wherein the second set of one or more electrodes is identical to the first set of one or more electrodes.

7. The system of claim 3, wherein the second set of one or more electrodes includes at least one electrode not included in the first set of one or more electrodes.

8. The system of claim 1, wherein the processing facility is further configured to designate the identified current level as a stapedius reflex threshold associated with the first set of one or more electrodes.

9. The system of claim 1, wherein the second stapedius reflex measurement session is temporally separated from the first stapedius reflex measurement session by at least one other intervening stapedius reflex measurement session.

10. The system of claim 1, wherein the first mapping data set comprises a default mapping data set.

11. The system of claim 1, wherein the processing facility is configured to automatically generate the second mapping data set as a function of the identified current level.

12. The system of claim 1, further comprising a storage facility configured to maintain the first and second mapping data sets.

13. The system of claim 1, wherein the processing facility is further configured to present, within a graphical user interface, data representative of at least one of the first mapping data set, the second mapping data set, and the identified current level.

14. The system of claim 1, wherein:
the detection facility is further configured to detect a frequency of the acoustic signal; and
the processing facility is further configured to use the frequency of the acoustic signal to designate one or more electrodes for inclusion in the first set of one or more electrodes.

15. A sound processor comprising:
an electrical stimulation management facility configured to adjust a current level of electrical stimulation applied by a cochlear implant associated with a patient by way of a first set of one or more electrodes in accordance with a sound level of an acoustic signal provided by a middle ear analyzer communicatively coupled to the sound processor by way of an interface system by directing the cochlear implant to incrementally increase the current level of the electrical stimulation in response to the middle ear analyzer incrementally increasing the sound level of the acoustic signal a stapedius reflex that occurs in response to the electrical stimulation is detected; and
a storage facility communicatively coupled to the electrical stimulation management facility and configured to store data representative of a current level at which the stapedius reflex occurs;
wherein the electrical stimulation management facility is further configured to utilize the stored data to determine one or more most comfortable stimulation levels associated with the set of one or more electrodes for use in one or more stimulation programs.

16. The sound processor of claim 15, wherein the electrical stimulation management facility is further configured to generate one or more user-selectable stimulation programs that utilize the one or more most comfortable stimulation levels.

17. A system comprising:
a middle ear analyzer configured to generate and transmit an acoustic signal;
a cochlear implant system configured to apply electrical stimulation having a current level based on a sound level of the acoustic signal by way of one or more electrodes implanted within a patient; and
an interface system communicatively coupled to the middle ear analyzer and to the cochlear implant system and configured to
receive the acoustic signal transmitted by the middle ear analyzer,
detect the sound level of the acoustic signal, and
use the sound level to control the current level of the electrical stimulation applied by the cochlear implant system;
wherein the middle ear analyzer is further configured to incrementally increase, during a first stapedius reflex measurement session, the sound level of the acoustic signal until a stapedius reflex that occurs in response to the cochlear implant system applying the electrical stimulation by way of a set of one or more electrodes implanted within the patient is detected; and
wherein the interface system is further configured to
synchronize the middle ear analyzer with the cochlear implant system during the first stapedius reflex measurement session in accordance with a first mapping data set representative of an association between a plurality of sound levels and a plurality of current levels,
identify a current level of the electrical stimulation at which the stapedius reflex occurs, and
automatically generate a second mapping data set based on the identified current level for use during a second stapedius reflex measurement session subsequent to the first stapedius reflex measurement session.

18. The system of claim 17, wherein the interface system is configured to synchronize the middle ear analyzer with the cochlear implant system in accordance with the first mapping data set during the first stapedius reflex measurement session by:
dynamically translating the sound level into a series of increasing current level values in accordance with the first mapping data set as the sound level incrementally increases during the first stapedius reflex measurement session; and
directing the cochlear implant system to dynamically increase the current level of the electrical stimulation being applied by way of the set of one or more electrodes in accordance with the series of increasing current level values as the sound level incrementally increases during the first stapedius reflex measurement session.

19. The system of claim 17, wherein the cochlear implant system is further configured to:
store data representative of the identified current level; and
utilize the stored data to determine one or more most comfortable stimulation levels associated with the first set of one or more electrodes for use in one or more stimulation programs.

20. A method comprising:
receiving, by an interface system, an acoustic signal transmitted by a middle ear analyzer;
detecting, by the interface system, a sound level of the acoustic signal;
using, by the interface system, the sound level of the acoustic signal to control a current level of electrical stimulation applied by a cochlear implant system associated with a patient;
synchronizing, by the interface system, the middle ear analyzer with the cochlear implant system in accordance with a first mapping data set representative of an association between a plurality of sound levels and a plurality of current levels during a first stapedius reflex measurement session in which the middle ear analyzer incrementally increases the sound level of the acoustic signal until a stapedius reflex that occurs in response to the cochlear implant system applying the electrical stimulation by way of a first set of one or more electrodes implanted within the patient is detected;
identifying, by the interface system, a current level of the electrical stimulation at which the stapedius reflex occurs; and
automatically generating, by the interface system, a second mapping data set based on the identified current level for use during a second stapedius reflex measurement session subsequent to the first stapedius reflex measurement session.
